(19) United States
(12) Patent Application Publication
(10) Pub. No.: US 2026/0166184 A1
(43) Pub. Date: Jun. 18, 2026

(54) COMPOSITIONS AND METHODS OF TARGETING AND IMAGING PRO-INFLAMMATORY MICROGLIA WITH AB PEPTIDE AMINO ACID RESIDUES FOR V-DOMAIN BINDING OF RAGE

(71) Applicant: Neurosn, Inc., Tucson, AZ (US)

(72) Inventors: Gilbert R. Gonzales, Tucson, AZ (US); Nigel R. Stevenson, Sugar Hill, GA (US); Jaime Simón, Angleton, TX (US)

(21) Appl. No.: 19/376,118

(22) Filed: Oct. 31, 2025

Related U.S. Application Data
(60) Provisional application No. 63/715,286, filed on Nov. 1, 2024.

(51) Int. Cl. A61K 51/08

(52) U.S. Cl. CPC A61K 51/088; A61K 2121/00; A61K 2123/00

(57) ABSTRACT

The receptor for advanced glycation end-products (RAGE) is a multi-ligand receptor member of the immunoglobulin super family which is able to bind Aβ peptide and 3-sheet fibrils. It is expressed in endothelial cells, smooth muscle cells, microglia and neurons, and is implicated in the transport of Aβ through the blood-brain barrier (BBB), oxidative stress-mediated neurotoxicity, and adverse microglia inflammatory responses. The interaction between RAGE and its ligands is thought to result in pro-inflammatory gene activation. Enhanced levels of RAGE ligands in Alzheimer's disease are thought to contribute to the cause of this disorder. Embodiments of the invention use the RAGE multi-ligand site as an anchoring loci for a conversion electron emitting compound rather than as a receptor to intrinsically activate or block inflammation through the RAGE intracellular cascade through activation of the RAGE cytoplasmic tail (ctRAGE) and mammalian diaphanous 1 (DIAPH1).

### CROSS-REFERENCE TO RELATED APPLICATIONS

This application claims priority to, and the benefit of the filing date of, U.S. Provisional Application Ser. No. 63/715,286, filed Nov. 1, 2024, the disclosure of which is hereby incorporated by reference herein in its entirety.

### INCORPORATION BY REFERENCE STATEMENT

The Sequence Listing XML file, identified as “NSN-03US2,” having a file size of 18,212 bytes and created on Oct. 29, 2025, is incorporated by reference in its entirety as part of this application.

### TECHNICAL FIELD

The present invention is directed to methods and compositions for targeting pro-inflammatory microglia in the central nervous system of a subject, and more particularly, to methods and compositions for imaging and inducing apoptosis in pro-inflammatory microglia with a radionuclide conjugate to treat neurodegenerative diseases.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Alzheimer's disease (AD) produces a relentless decline of certain brain areas with resulting erosion of memory, the reduction the ability to perform tasks as well as affecting organizational abilities and creating poor judgment in the affected individual. The rate of progression from mild to moderate to severe AD can vary from person to person. The brain changes in AD can begin more than 20 years before the first symptoms appear. AD is associated with the presence of tangles and amyloid plaques. As tangles and amyloid plaques form in the brain, the areas of brain tissue that are affected become damaged and work less effectively. To date, most research has focused blocking or reducing amyloid plaque formation based on the amyloid cascade hypothesis. However, treatment for AD is still elusive despite the many compounds that have been tried in development and the billions of dollars expended to date.

Microglia are the resident phagocytes and innate immune cells of the brain. Investigators have recognized the importance of microglia in the homeostasis, as well as various pathologies, of the central nervous system. It is now widely accepted that clustered populations of reactive microglia are hallmarks of neurological disorders where neuro-inflammation is present and contributes to the mechanisms of neuronal damage. Subsets of reactive microglia called pro-inflammatory microglia are at least partially responsible for the cascade of events that lead to the deposition of amyloid beta (Ap), a substance that is toxic to neurons as it accumulates in amyloid plaques. These microglia have been shown to be associated with extracellular amyloid plaques and neurons containing tau pathology, hallmarks of neurodegenerative diseases like AD. Accumulation of hyper-phosphorylated tau in neurons is correlated with progressive cognitive dysfunction and neuronal loss. Furthermore, reactive microglia have been associated with a variety of neurodegenerative diseases including AD, Parkinson's disease (PD) and amyotrophic lateral sclerosis (ALS).

The radionuclide Sn-117m's conversion electrons are a unique therapeutic particle emission with an average energy of 0.14 MeV that interact with tissue. Unlike beta rays that are emitted by most therapeutic radioisotopes, the Sn-117m conversion electrons are mono-energetic and travel an absolute maximum distance in tissue of ˜300 μm. This limits the therapeutic effect to tissue targeted by Sn-117m without damaging adjacent healthy tissue. Additionally, it has been demonstrated that at very low (i.e., hormetic) radiation doses, well below conventional DNA-breaking doses, the conversion electrons have a positive therapeutic effect by inducing apoptosis in macrophages and microglia. Sn-117m's unique conversion electron energy has several distinct advantages over traditional radiation therapy including an ideal two-week half-life and an unprecedented safety profile which allows shipping with no special handling procedures. Sn-117m also emits gamma photons, which are similar in energy to that of ^{99m}Tc, that can be imaged with a standard γ-camera or single-photon emission computed tomography (“SPECT”). The gamma photons thus allow tissue targeted by Sn-117m to be imaged.

Accordingly, there is a need for a radiopharmaceutical compound configured to reliably induce apoptosis and/or image microglia. There is also a need for reliably producing said radiopharmaceutical compound.

Radioisotopes, such as Sn-117m, may be incorporated into a radiopharmaceutical compound using chelation. For many metal ions, the aqueous chemistry involved to attach the metal to the protein is relatively mild and the construct containing the carrier molecule is prepared first. In the last step, the isotope is added to the chelant containing construct to prepare the final radiopharmaceutical. This sequence, where the isotope is added as the last step in the formulation, is preferred. It is sometimes called a “one-step” approach because the isotope is only handled one time. However, in some cases, the chemistry associated with attaching the metal ion to the chelating agent requires harsh conditions. Attaching metal ions with high oxidation states in some cases requires harsh conditions. For example, Sn-117m produced in high specific activity is prepared as the stannic, Sn(IV), ion. Forming Sn-117m chelates with a DOTA (1,4,7,10-Tetraazacyclododecane-1,4,7,10-tetraacetic acid) chelating agents requires temperatures greater than or equal to 140° C. This high temperature is usually too high when the carrier molecules are peptides or antibodies because the amide linkages and hydrogen bonding that is essential for activity is compromised. For metals such as Sn(IV), the final product has to be prepared by first combining the metal with the chelating agent. In a second step, the resulting chelate is attached to the carrier molecule. This is sometimes referred to as a “two-step” approach. It is less desirable because the radioisotope must be handled two times and typically, yields are lower than with a one-step approach. Accordingly, there is a need for producing a radiopharmaceutical compound configured to reliably induce apoptosis and/or image microglia using a one-step approach.

Click chemistry is an approach to chemical synthesis that emphasizes efficiency, simplicity, selectivity, and modularity in chemical processes used to join molecular building blocks. It includes both the development and use of “click reactions”, a set of simple, biocompatible chemical reactions that meet specific criteria like high yield, fast reaction rates, and minimal byproducts. Generally speaking, click chemistry reactions have a sufficiently high modularity, insensitivity to solvent parameters, high chemical yield, insensitivity to oxygen and water, regiospecificity, stereospecificity, and a large thermodynamic driving force (e.g., greater than or equal to 20 kcal/mol). Accordingly, there is a need for applying click chemistry reactions to reliably create therapeutic compounds configured to reliably induce apoptosis and/or image microglia.

### SUMMARY

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

Unlike the failed approach to treating AD through the “amyloid cascade hypothesis”, embodiments of the present invention are directed to methods of treating AD through the induction of apoptosis (programmed cell death) in pro-inflammatory microglia of the brain. This therapeutic approach is based on the “neuro-inflammatory hypothesis” as well as the “microglial dysfunction hypothesis”. These hypotheses hold that microglia that reside in the brain are at least partially responsible for the cascade of events that lead to the deposition of amyloid beta (AB), a substance that is toxic to the brain as it accumulates and is responsible for neuronal cell death. Microglia have also been shown to be associated with intra-neuronal tau pathology in the brain and tau is correlated with progressive cognitive dysfunction and neuronal loss. Embodiments of the present invention utilize radionuclide conjugates having a unique therapeutic energy form called conversion electrons (CE) that has been shown in experimental models to induce apoptosis in radiation doses that are extremely low. These CE doses are below typical therapeutic radiation DNA fracturing doses that lead to necrotic cell death such as used in cancer treatments. A CE compound has already been successfully delivered intravenously into the brain in humans, although not for the purposes of targeting pro-inflammatory microglia to treat neurodegenerative diseases like AD, PD, and ALS.

And so, one aspect of the present invention is directed to a radiopharmaceutical compound configured to induce apoptosis, image, or both induce apoptosis and image pro-inflammatory microglia in the central nervous system of a subject. Such a radiopharmaceutical compound may include (1) a targeting agent, T_{A}, wherein the targeting agent is configured to bind with the RAGE receptor, (2) a chelated radionuclide, Rad(C), comprising a chelating agent, C, and a radionuclide, Rad, wherein the radionuclide is included in an effective amount to induce apoptosis, image, or both induce apoptosis and image pro-inflammatory microglia; and (3) a spacing moiety, S_{M}, wherein the spacing moiety is conjugated with the targeting agent, and wherein the spacing moiety reduces steric hinderance for the radiopharmaceutical compound when compared to otherwise identical radiopharmaceutical compounds not including the spacing moiety. Further, the inclusion of the spacing moiety results in a water soluble radiopharmaceutical compound, (where an otherwise identical radiopharmaceutical compound not including the spacing moiety would not be water soluble).

Another aspect of the present invention is directed to a method of inducing apoptosis in pro-inflammatory microglia comprising administering a radiopharmaceutical compound (such as that described in the first aspect of the invention) to a subject in an effective amount to induce apoptosis.

Another aspect of the present invention is directed to a method of synthesizing a radiopharmaceutical compound configured to induce apoptosis, image, or both induce apoptosis and image pro-inflammatory microglia in the central nervous system of a subject. The method includes (1) chelating a radionuclide, Rad, with a chelating agent, C, to form a chelated radionuclide, Rad(C), (wherein chelating comprises dissolving a radionuclide and a chelating agent together in a solvent under mild conditions to form a dissolved mixture), and (2) conjugating the chelating agent or chelated radionuclide with a substituent including a targeting agent, T_{A}.

Another aspect of the present invention is directed to a radiopharmaceutical compound containing a targeting moiety and an isotope wherein the targeting moiety targets amyloid-beta peptide receptors.

### BRIEF DESCRIPTION OF THE DRAWINGS

The accompanying drawings, which are incorporated in and constitute a part of this specification, illustrate embodiments of the invention and, with a detailed description of the embodiments given below, serve to explain the principles of the invention.

FIG. 1 is a graph showing HPLC data from radioactive species.

FIG. 2 is a graph showing HPLC data from all detected species.

FIG. 3 is an overlay chromatogram (HPLC data) of DOTA, Polypeptide-Azide, and DOTA-Polypeptide conjugate.

FIG. 4 shows results of thin layer chromatography (TLC), 2-hour incubation; DOTA-Lu-177 pH 6.0 and 6.5, and Lu Control; Mobile phase: Sodium Citrate.

FIGS. 5, 6, and 7 are chromatograms of DOTA-Lu-177 pH 6.5 (FIGS. 5 and 6) and free-Lu-177 as a control (FIG. 7).

FIG. 8 is an iTLC (instant thin layer chromatography) comparison showing the product remaining at the origin (left panel) and free Lu migrating with the solvent front (right panel).

FIGS. 9 and 10 are radiometric chromatograms showing conjugated DOTA-Lu-Polypeptide (FIG. 9) and free Lu-177 (FIG. 10).

FIG. 11 is a radiometric chromatogram of conjugation.

FIGS. 12 and 13 shows an example of the click chemistry reaction to form a conjugate radiopharmaceutical compound, with FIG. 12 showing the reaction, and FIG. 13 showing the two stereoisomer products.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the invention are directed to methods of inducing apoptosis of, imaging, or both inducing apoptosis of and imaging pro-inflammatory microglia in the central nervous system of a subject. The methods may treat neurodegenerative diseases in which pro-inflammatory microglia play a role in the underlying disease process by disrupting the neuro-inflammatory cascade that is implicated in these disorders. Exemplary neurodegenerative diseases that may be treated include AD, PD, and ALS. As used herein, “pro-inflammatory microglia” means microglia that express the receptor for advanced glycation end-products (RAGE) at a higher level than regular microglia.

The methods utilize a radiopharmaceutical compound includes a radionuclide conjugate, that includes a radionuclide, (“Rad” such as Sn-117m or [Sn-117m]²⁺, or lutetium-177, Lu-177), and a targeting agent (T_{A}) capable of binding RAGE receptors on pro-inflammatory microglia (e.g., Rad-T_{A}). In one embodiment, the radiopharmaceutical (e.g., Sn-117m(DOTA)-T_{A}) includes a chelated radionuclide, Rad(C) (e.g., Sn-117m(DOTA)), wherein radionuclide is chelated using a chelating agent (e.g., DOTA). In further embodiments, the radiopharmaceutical compound further includes a linker moiety (L_{M}). In one such embodiment, the chelated radionuclide is attached to the targeting agent via the linking moiety (e.g., Rad(C)-L_{M}-T_{A}). In another embodiment, the radiopharmaceutical compound further includes a spacer moiety (S_{M}). In one such embodiment, the spacer moiety is positioned between the targeting agent and the chelated radionuclide (e.g., Rad(C)-S_{M}-T_{A} or Rad(C)-L_{M}-S_{M}-T_{A}). Further embodiments of the invention are directed to methods of producing a radionuclide conjugate that is capable of targeting RAGE receptors on pro-inflammatory microglia. Additional embodiments of the invention are directed to radiopharmaceutical compositions that include radionuclide conjugates that are capable of targeting RAGE receptors on pro-inflammatory microglia. The radionuclide conjugate is capable binding to the RAGE receptor and results in inhibition of pro-inflammatory microglia, which is believed to occur without internal cellular triggering of the cytoplasmic tail of RAGE and the intracellular effector, diaphanous-1.

The radionuclide (R) is an isotope capable of producing a conversion election (CE). In an exemplary embodiment, the radionuclide is Sn-117m. In an embodiment, Sn-117m has at least a medium specific activity, i.e., an activity of at least 100 Ci/g. In another embodiment, the Sn-117m has a specific activity that is at least a high specific activity, i.e., an activity of at least 1,000 Ci/g. In yet another embodiment, the Sn-117m has a specific activity that is a very high specific activity, i.e., a specific activity of at least 10,000 Ci/g. For purposes of the present invention, the range for the specific activity of Sn-117m are defined as follows:
Low Specific Activity Sn-117m: <100 Ci/g; such as manufactured with reactors;
Medium Specific Activity Sn-117m: 100-1,000 Ci/g; such as manufactured with proton accelerators but low yield;
High Specific Activity Sn-117m: 1,000-10,000 Ci/g; such as manufactured with proton accelerators (but higher Sn-113 so limited use); primarily manufactured with alpha accelerators; and
Very High Specific Activity Sn-117m: >10,000 Ci/g; such as manufactured with alpha accelerators

The radionuclide to be used in the aspects of the present invention is not limited to Sn-117m, and thus may be other radionuclides, such as Lu-177, or any other radionuclide that could be considered useful (such as therapeutically useful) in the conjugates described herein. Such other radionuclides may include, but are not limited to, Zr-89, Lu-177, Ac-225, Pb-212, In-111, Zr-89, Ga-68, Ga-67, Sm-153, Ra-223, and Tb-161.

The radionuclide may be chelated using a chelating agent (i.e., chelant) to define a chelated radionuclide (“Rad(C)”). In embodiments of the invention, the radionuclide is conjugated to the targeting agent with a chelating molecule, such as aminobenzyl DOTA (ABD), which the amine group on ABD is converted to an isothiocyanate group to form isothiocyanatebenzyl DOTA (IBD), or directly with diethylene triamine pentaacetic acid (DTPA). Other chelants may be used. In some embodiments, the chelating agent is modified to be more easily conjugated with another component of the radiopharmaceutical composition (described further below). In some embodiments, the radionuclide is chelated in an aqueous solution. In other embodiments, the radionuclide is chelated without using water or an aqueous solution (i.e., a non-aqueous process).

In some embodiments, the radionuclide is chelated in a non-aqueous solution. Not to be limited by theory, it is believed that the reason harsh conditions are needed to form chelates in aqueous solutions is due to formation of insoluble hydroxide metal species. Chelating agents such as DOTA have both amines and carboxylic acid functionality that if they exist in the protonated form make it difficult to form metal complexes. Therefore, when in aqueous solution, raising the pH to a range where both amines and carboxylic acids are deprotonated is conventionally done to achieve chelation. Unfortunately, the solubility of certain metal ions in aqueous solutions decreases as the pH is raised due to reactions with hydroxide ions. Accordingly, the aim of the non-aqueous process for chelation is to perform a chelation reaction without the presence of hydroxide ions so that the metal ions will remain in solution and it may be possible to form chelates under milder conditions.

In such embodiments, the combination of chelating agent and radioactive metal isotope is important. For example, Sn-117m readily forms complexes with DTPA, DOTA, ADB, and IDB in non-aqueous solutions. In one embodiment, Sn-117m readily forms chelates with DTPA, DOTA, ADB, or IDB under mild conditions (e.g., non-acidic environments at temperatures between 20° C. and 60° C.). This differs drastically from harsh environments, wherein the pH can be less than or equal to 5 and the temperature may exceed 140° C. However, the complexes are relatively unstable and may fall apart in the bloodstream of a patient. In contrast, it takes harsh conditions to form the complex between Sn-117m and DOTA in water. However, once formed the complexes are relatively very stable. In some embodiments, non-aqueous solvents that have the capability to dissolve both the desired metal ion and the targeting molecule. A non-limiting list of such non-aqueous solvents includes hydrocarbons, halogenated hydrocarbons, ethers, alcohols, glycols, and polyols. In a one embodiment, the non-aqueous solvent is selected from a list consisting of tetrahydrofuran (THF), dimethyl sulfoxide (DMSO), dimethylformamide (DMF), acetonitrile, acetone, 1,2-dimethoxyethane, ethanol, ethylene glycol, glycerin, hexamethylphosphoroustriamide, methanol and pyridine. In a preferred embodiment, the non-aqueous solvent is selected from a list consisting of DMSO, THF and DMF.

In some embodiments involving the use of a non-aqueous solvent for chelating the radionuclide, a specific chelating agent may be used. In one such embodiment, the chelating agent is an aminocarboxylic acid. In a further embodiment thereof, the chelating agent is an aminocarboxylic acid selected from the list consisting of nitrilotriacetic acid (NTA), ethylenediaminetetraacetic acid (EDTA), diethylenetriaminepentaacetic acid (DTPA), 1,4,7,10-Tetraazacyclododecane-1,4,7,10-tetraacetic acid (DOTA), aminobenzyl DOTA (ABD), isothiocyanatebenzyl DOTA (IBD), other similarly suitable chelating agents, and suitable derivatives thereof.

In some embodiments, the chelating agent is a bifunctional chelating agent such that it contains both the chelant functionality plus a functional group to attach to a carrier molecule (e.g., a linker moiety or a first linker moiety portion as discussed further below). In one embodiment, the bifunctional chelating agent is selected from the list consisting of p-SCN-Bn-TCMC, p-NO2-Bn-Cyclen, p-SCN-Bn-HEHA, DOTAM-mono-acid, p-NO2-Bn-DOTA, p-NH₂-Bn-DOTA, p-SCN-Bn-DOTA, p-NH₂-Bn-DOTA-tetra(t-Bu ester), Maleimido-mono-amide-DOTA, Butyne-DOTA-tris(t-butyl ester), 2-Aminoethyl-mono-amide-DOTA-tris(t-Bu ester), p-SCN-Bn-DTPA, p-SCN-Bn-CHX-A″-DTPA, Maleimido-mono-amide-DTPA, p-NH₂-Bn-PCTA, p-SCN-Bn-PCTA, NO2A-Butyne, p-SCN-Bn-NOTA, p-SCN-Bn-Deferoxamine, Deferoxamine-maleimide, other suitable bifunctional chelating agents, and suitable derivatives thereof. A list of bifunctional chelating agents can be found in the Macrocyclics web site (https://www.macrocyclics.com/online-catalog/bifunctional-chelators-bfcs/) and are incorporated herein by reference.

The non-aqueous process for preparing a chelated radionuclide includes a dissolution step and may involve one or more of the following steps: a base treatment step, a heat treatment step, and a purification step. In one embodiment, the non-aqueous process for preparing a chelated radionuclides includes all of the steps above. In one embodiment, the non-aqueous process for preparing a chelated radionuclide does not include a base treatment step. In one embodiment, the non-aqueous process for preparing a chelated radionuclide does not include a heat treatment step. In one embodiment, the non-aqueous process for preparing a chelated radionuclide does not include a purification step.

With regard to the dissolution step for preparing a chelated radionuclide, the dissolution step involves dissolving the chelating agent and the radionuclide in a non-aqueous solution to form a dissolved mixture. In one such embodiment of the dissolution step, the chelating agent and the radionuclide are dissolved in a non-aqueous solution before conjugating the chelating agent with any other portions of a radiopharmaceutical compound such as a linking moiety, spacing moiety, or targeting agent. In a preferred embodiment, prior to dissolution, the radiopharmaceutical compound is complete but for the radionuclide not yet being complexed in the chelating agent (e.g., C-T_{A}, C-S_{M}-T_{A}, C-L_{M}-S_{M}-T_{A}, C-L_{M}-T_{A}, etc.). Alternatively, the dissolution step may involve dissolving a conjugated chelating agent. In one such embodiment, the chelating agent is conjugated with a linking moiety prior to dissolution, wherein the linking moiety may optionally be conjugated with a spacing moiety or a targeting agent. In an alternate embodiment, the dissolution step involves dissolving chelating agent that is conjugated with a linking moiety portion (e.g., a first linking moiety portion) prior to dissolution. In an alternate embodiment, the dissolution step involves dissolving a chelating agent that is conjugated with a spacing moiety, wherein the spacing moiety may optionally be conjugated with a targeting agent. In an alternate embodiment, the dissolution step involves dissolving a chelating agent that is conjugated with a targeting agent. In the dissolution step, the chelating agent and the radionuclide may be added in a 1:1 proportion, or alternatively 2:1 chelating agent to radionuclide, alternatively 5:1 chelating agent to radionuclide, alternatively 10:1 chelating agent to radionuclide, alternatively 20:1 chelating agent to radionuclide; alternatively 50:1 chelating agent to radionuclide, or 100:1 chelating agent to radionuclide. In embodiments where an excess of chelating agent is added, the process may involve an additional chelating agent recovery step.

With regard to embodiments of the non-aqueous process including the base treatment step, base is added to the dissolved mixture in an effective amount to scavenge hydrogen ions or hydronium ions formed. The base may be selected from a hydroxide salt, a hydroxide solution, a carbonate salt, a carbonate solution, a bicarbonate salt, a bicarbonate solution, a nitrite salt, a nitrite solution, other bases having similar strengths, or a combination thereof. In one embodiment, a solid base is added to the dissolved mixture. In one embodiment, the solid base may be selected from a list consisting of sodium carbonate and potassium carbonate. In one embodiment, the base treatment step is configured to add a sufficient amount of base in order to match or exceed the expected amount of hydrogen ions formed in the dissolution step. In one such embodiment, the expected amount of hydrogen ions may be calculated using the reaction formula specific to the chelating agent used.

With regard to embodiments including the heat treatment step, the dissolved mixture can be heated to a heat treatment temperature for a heat treatment duration, wherein the heat treatment temperature and the heat treatment duration are corelated variables (i.e., a higher temperature may result in a lower necessary duration). The heat treatment duration can be greater than or equal to 1 hour, alternatively greater than or equal to 2 hours, alternatively greater than or equal to 4 hours, alternatively greater than or equal to 6 hours, alternatively greater than or equal to 12 hours, alternatively greater than or equal to 24 hours, alternatively greater than or equal to 48 hours, or alternatively greater than or equal to 72 hours. In one embodiment, the heat treatment duration is 12 hours. The heat treatment duration may depend on the heat treatment temperature. In one embodiment, the heat treatment temperature is greater than or equal to room temperature and less than or equal to 60° C. In one embodiment, the heat treatment temperature is 60° C. In a further embodiment thereof, the heat treatment duration is 12 hours.

In embodiments not including the heat treatment step, the dissolved mixture may optionally be subjected to an incubation step instead wherein the dissolved mixture is incubated at room temperature for an incubation duration. In such embodiments, the incubation duration may be greater than or equal to 12 hours, alternatively greater than or equal to 24 hours, alternatively greater than or equal to 48 hours, or alternatively greater than or equal to 72 hours.

With regard to the purification step, the chelated radionuclide may be separated from the non-aqueous solvent. In one embodiment, purification of the chelated radionuclide is accomplished by traditional methods such as chromatographic procedures, filtration (e.g., nanofiltration, ultrafiltration), evaporation of the solvent, or other suitable separation techniques. In one embodiment, the purification step involves a chromatographic procedure that isolates the chelated radionuclide in an aqueous solution.

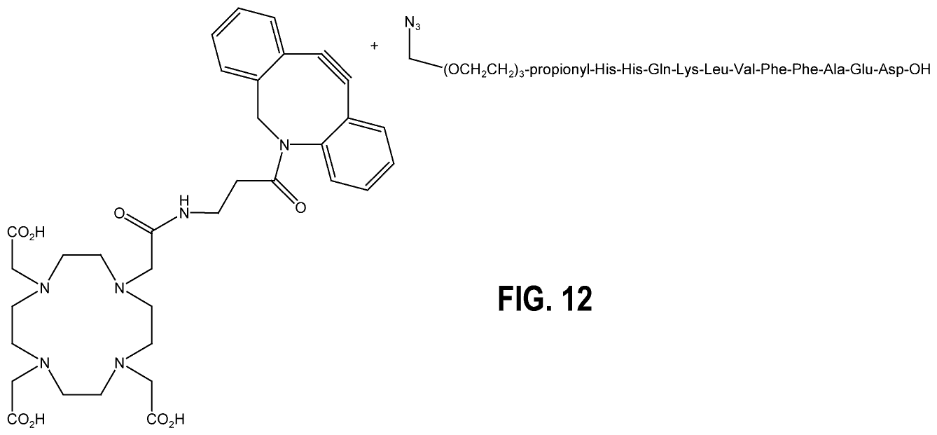

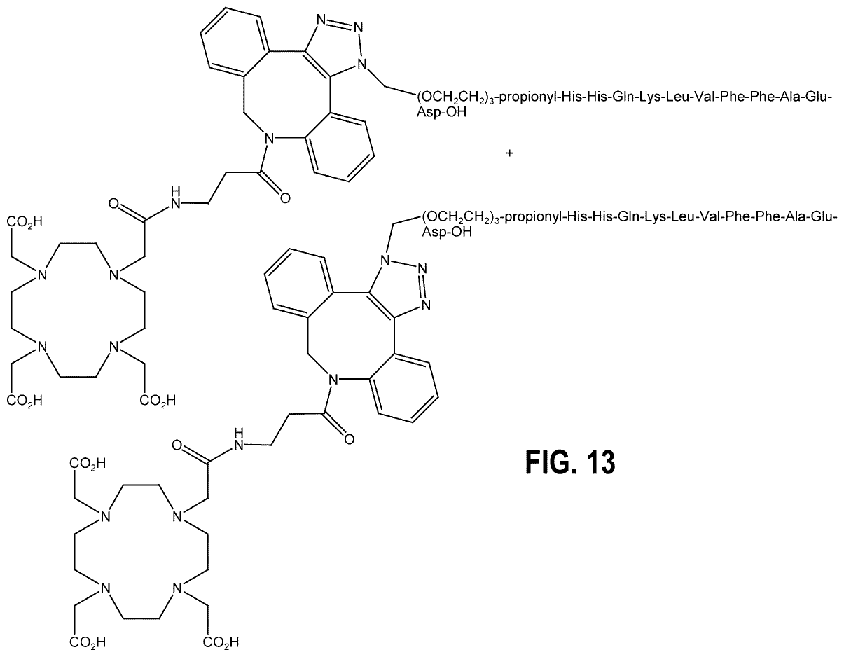

The radionuclide is conjugated with a targeting agent (T_{A}). In embodiments of the invention, the targeting agent includes a truncated portion of the Aβ peptide amino acid chain, which has been identified as critical in binding to RAGE receptors expressed by pro-inflammatory microglia. In one embodiment, the targeting agent includes a portion of the Aβ peptide amino acid chain having at least the 7 amino acid length between at least the seventeenth and twenty-third amino acid (discussed further below), or alternatively having at least the 8 amino acid length between the sixteenth and twenty-third amino acid (discussed further below). In a further embodiment thereof, the truncated portion of the Aβ peptide amino acid chain is configured to be positioned distally from the radionuclide in the radiopharmaceutical compound. The RAGE receptor is also found at the blood brain barrier and is responsible for active transport of Aβ from blood circulation to the brain. In embodiments of the invention, the targeting agent is an 8-amino acid chain Aβ(16-23) having the amino acid sequence KLVFFAED (SEQ ID NO 1) or an amino acid chain Aβ(23-17) (modified with a terminal lysine (K)) having amino acid sequence KDEAFFVL (SEQ ID NO. 2). The amino acid chain Aβ(23-17) (modified with a terminal lysine (K)) having amino acid sequence KDEAFFVL (SEQ ID NO. 2) may also be referred to herein as K-Aβ(23-17). Alternatively, the amino acid chain Aβ(23-17) may be modified with a terminal alanine (A) to define an amino acid sequence ADEAFFVL (SEQ ID NO. 3) and may be referred to herein as A-Aβ(23-17). In one embodiment of the invention, the radionuclide is conjugated to a one or more targeting agents selected from the list consisting of Aβ(16-23) having the amino acid sequence KLVFFAED (SEQ ID NO. 1), K-Aβ(23-17) having amino acid sequence KDEAFFVL (SEQ ID NO. 2), and A-Aβ(23-17) having amino acid sequence ADEAFFVL (SEQ ID NO. 3), or some mixture thereof. In embodiments of the invention may include a charge neutralizing blocking molecule at either end of the targeting agent, such as at the one or both ends of the Aβ fragments identified above. An embodiment of a conjugate including the targeting agent KLVFFAED (SEQ ID NO. 1)—and the production of same—is shown in FIGS. 12 and 13. In other embodiments, the targeting moiety may include Azido-PEG3-His-His-Gln-Lys-Leu-Val-Phe-Phe-Ala-Glu-Asp-OH.

In some embodiments, longer versions of these Aβ fragments may be used to decrease steric hinderance that may be caused by the radionuclide/chelated radionuclide. In one embodiment wherein the steric hinderance is sufficiently reduced by using a longer version of the Aβ fragments, the resulting radiopharmaceutical composition is water soluble. In one such embodiment, an additional 3 amino acids from the Aβ peptide amino acid chain are included, resulting in an 11-amino acid chain Aβ(13-23) HHQKLVFFAED (SEQ ID NO. 4) or Aβ(26-16) SGVDEAFFVLK (SEQ ID NO. 5). In a further embodiment thereof, each of the above 11-amino acid chains may be modified to include a terminal lysine, resulting in 12-amino acid chains K-Aβ(13-23) KHHQKLVFFAED (SEQ ID NO. 6) or K-Aβ(26-16) KSGVDEAFFVLK (SEQ ID NO. 7). In a preferred further embodiment thereof, each of the above 11-amino acid chains may be modified to include a terminal alanine, resulting in 12-amino acid chains A-Aβ(13-23) AHHQKLVFFAED (SEQ ID NO. 8) or A-Aβ(26-16) ASGVDEAFFVLK (SEQ ID NO. 9). In one embodiment, it is preferable to add a terminal alanine instead of a terminal lysine because the terminal alanine only has one amine group that can be used for conjugation whereas the terminal lysine has two amine groups that could be used for conjugation, leading to two alternative products or structures. In some embodiments, the terminal lysine or the terminal alanine of the 12-amino acid chain is configured to conjugate with the linking moiety (discussed further below). In one embodiment, a targeting agent having a 12-amino acid chain, including the terminal lysine group or the terminal alanine, enables the resulting radiopharmaceutical composition to omit a spacing moiety (discussed further below) while sufficiently reducing steric hinderance and enabling the resulting radiopharmaceutical compound to be water soluble.

In some embodiments, the radiopharmaceutical compound includes a spacer moiety or spacing moiety (S_{M}). In one such embodiment, targeting agent is conjugated with the spacer moiety (S_{M}-T_{A}). In one such embodiment, the spacer moiety is conjugated with both the targeting agent to the chelated radionuclide (Rad(C)-S_{M}-T_{A}). In an alternate such embodiment, the spacer moiety is conjugated with both the targeting agent to the linking moiety (Rad(C)-L_{M}-S_{M}-T_{A}) (discussed further below). In some embodiments wherein the spacing moiety is conjugated with the targeting agent, the steric hinderance caused by the radionuclide/chelated radionuclide is reduced. In one embodiment wherein the steric hinderance is sufficiently reduced by conjugating the spacing moiety to the targeting agent, the resulting radiopharmaceutical composition is water soluble.

The spacing moiety may be a chain of amino acids. In one embodiment, the spacing moiety is a chain of amino acids that is not a truncated portion of the Aβ peptide amino acid chain. In one such embodiment, the spacing moiety is a chain of amino acids having a length greater than or equal to 2 amino acids and less than or equal to 6 amino acids, or alternatively having a length greater than or equal to 3 amino acids and less than or equal to 5 amino acids. In one embodiment, the spacing moiety is a chain of amino acids having a length equal to 2 amino acids, alternatively equal to 3 amino acids, alternatively equal to 4 amino acids, alternatively equal to 5 amino acids, or alternatively equal to 6 amino acids. In one embodiment, the spacing moiety is an amino acid chain comprising a terminal lysine. In some embodiments, the terminal lysine of the spacing moiety is configured to conjugate with the linking moiety (discussed further below). In one embodiment, the spacing moiety is a 4-amino acid chain comprising a terminal lysine, including but not limited to KHHG (SEQ ID NO. 10). In a preferred embodiment, the spacing moiety is a 4-amino acid chain comprising a terminal alanine, including but not limited to AHHG (SEQ ID NO. 11). In one embodiment, the spacing moiety is conjugated with an 8-amino acid chain targeting agent derived from the Aβ peptide disclosed above, resulting in 12-amino acid chains including: S_{M}-Aβ(16-23), resulting in KHHGKLVFFAED (SEQ ID NO. 12) or AHHGKLVFFAED (SEQ ID NO. 13); S_{M}-Aβ(23-16), resulting in KHHGDEAFFVLK (SEQ ID NO. 14) or AHHGDEAFFVLK (SEQ ID NO. 15); the terminal lysine modified version of K-Aβ(23-17), S_{M}-K-Aβ(23-17), resulting in KHHGKDEAFFVL (SEQ ID NO. 16) or AHHGKDEAFFVL (SEQ ID NO. 17); and the terminal alanine modified version of A-Aβ(23-17), S_{M}-A-Aβ(23-17), resulting in KHHGADEAFFVL (SEQ ID NO. 18) or AHHGADEAFFVL (SEQ ID NO. 19).

In embodiments of the invention, the radionuclide conjugate includes a linking moiety between the chelating agent and the targeting agent. The linking moiety may function to allow a binding site between the chelating agent and the targeting agent. The linking moiety may also provide relief from steric hinderance that the chelating agent may cause with respect to the binding of the targeting agent and the RAGE receptor. In embodiments of the invention, the linking moiety may include an alkyl group containing chain having between 2 and 10 carbons, an ether group containing chain having between 1 and 10 ether groups, or other chains of subunits capable of relieving steric hinderance between the chelating agent and the binding of the targeting agent with the RAGE receptor. It will be appreciated that the alkyl group containing chain and ether group containing chain may include other components, such as alkyl rings, aromatic rings, amide groups, amino groups, hydroxyl groups, etc.

In some embodiments, the linking moiety may comprise a first linking moiety portion (L_{P1}) and a second linking moiety portion (L_{P2}), such that the first linking moiety portion and the second linking moiety portions are configured to conjugate preferentially with each other to form the linking moiety (e.g., R-L_{P1}+L_{P2}-R′→R-L_{M}-R′). In such embodiments, the first linking moiety portion is conjugated with the chelated radionuclide (Rad(C)-L_{P1}) and the second linking moiety portion is conjugated with the at least one of the targeting agent (L_{P2}-T_{A}) and/or the spacing moiety (L_{P2}-S_{M}). In one embodiment, the second linking moiety portion is conjugated with the targeting agent. In one embodiment, the second linking moiety portion is conjugated with the spacing moiety. In a further embodiment thereof, the spacing moiety is further conjugated with the targeting agent (L_{P2}-S_{M}-T_{A}).

In a preferred embodiment, the first linking moiety portion and the second linking moiety portion are configured to react using a click chemistry reaction such that the first linking moiety portion comprises a first click chemistry portion and the second linking moiety portion comprises a second click chemistry portion, wherein the first click chemistry portion and the second click chemistry portion preferentially react to form the linking moiety. In some embodiments, the first click chemistry portion and the second click chemistry portion form a pair configured to enable a click chemistry reaction.

In one embodiment, the first click chemistry portion and the second click chemistry portion form a pair configured to enable a strain-promoted azide alkyne cycloaddition (SPAAC) reaction. In one such embodiment, the first click chemistry portion comprises a terminal azide and the second click chemistry portion comprises a cyclooctyne. In another embodiment, the first click chemistry portion comprises a cyclooctyne and the second click chemistry portion comprises a terminal azide. When used, a cyclooctyne may be selected from the list consisting of a difluorooctyne (DIFO) such as 3-difluorooctyne, a dibenzylcyclooctyne (DIBO), an azadibenzylcyclooctyne (ADIBO) such as 5-azadibenzylcyclooctyne, and a cylcopropanecyclooctyne (CPO) such as 5,6-cylcopropanecyclooctyne. However, other cyclooctynes may be used including but not limited to DBCO compounds available from Vector Laboratories, available at https://vectorlabs.com/product-category/cross-linkers-and-dspe-linkers/click-chemistry/dbco.reagents and incorporated by reference herein, including but not limited to DBCO-triethylene glycol-N-Hydroxysuccinimide(NHS) ester, DBCO-tetraethylene glycol-NHS ester, DBCO-pentaethylene glycol-NHS ester, DBCO-carboxylic acid, DBCO-amine, DBCO-triethylene glycol-amine, DBCO-tetraethylene glycol-amine, DBCO-pentaethylene glycol-amine, or any other suitable compound having a cyclooctyne ring, may be used. When a terminal azide is used, it may be selected from the list consisting of azido-triethylene glycol-NHS ester, azido-tetraethylene glycol-NHS ester, azido-pentaethylene glycol-NHS ester, azido-triethylene glycol-maleimide, azido-tetraethylene glycol-maleimide, azido-pentaethylene glycol-maleimide, azido-triethylene glycol-amine, azido-tetraethylene glycol-amine, azido-pentaethylene glycol-amine. However, any other azide reagent available from vector laboratory, available at https://vectorlabs.com/product-category/cross-linkers-and-dspe-linkers/click-chemistry/azide-reagents and incorporated by reference herein, may be used instead. Other types of click chemistry may be useful in these embodiments including, but not limited to, strain-promoted alkyne-nitrone cycloaddition (SPANC), copper(I)-catalyzed azide-alkyne cycloaddition (CuAAC), or any other suitable click chemistries.

The radionuclide conjugates of Sn-117m with the above identified targeting agents(e.g., Aβ(16-23) KLVFFAED (SEQ ID NO. 1), K-Aβ(23-17) KDEAFFVL (SEQ ID NO. 2), A-Aβ(23-17) ADEAFFVL (SEQ ID NO. 3), K-Aβ(13-23) KHHQKLVFFAED (SEQ ID NO. 6), K-Aβ(26-16) KSGVDEAFFVLK (SEQ ID NO. 6), A-Aβ(13-23) AHHQKLVFFAED (SEQ ID NO. 8), or A-Aβ(26-16) KSGVDEAFFVLK (SEQ ID NO. 6.2)) and the radionuclide conjugates of Sn-117m with the combined spacing moieties and targeting agents (e.g., S_{M}-Aβ(16-23) KHHGKLVFFAED (SEQ ID NO. 12) and AHHGKLVFFAED (SEQ ID NO. 13), S_{M}-Aβ(23-16) KHHGDEAFFVLK (SEQ ID NO. 14) and AHHGDEAFFVLK (SEQ ID NO. 15), S_{M}-K-Aβ(23-17) KHHGKDEAFFVL (SEQ ID NO. 16) or AHHGKDEAFFVL (SEQ ID NO. 17), and S_{M}-A-Aβ(23-17) KHHGADEAFFVL (SEQ ID NO. 18) or AHHGADEAFFVL (SEQ ID NO. 19)) can cross the intact or damaged blood brain barrier bind to RAGE receptors on pro-inflammatory microglia to induce apoptosis in the pro-inflammatory microglia via conversion electrons, thereby reducing the deposition of Aβ, and suppressing the propagation of hyper-phosphorylated tau protein in the brain. Similarly, the radionuclide conjugates of Lu-177 with the above identified targeting agents(e.g., Aβ(16-23) KLVFFAED (SEQ ID NO. 1), K-Aβ(23-17) KDEAFFVL (SEQ ID NO. 2), A-Aβ(23-17) ADEAFFVL (SEQ ID NO. 3), K-Aβ(13-23) KHHQKLVFFAED (SEQ ID NO. 6), K-Aβ(26-16) KSGVDEAFFVLK (SEQ ID NO. 6), A-Aβ(13-23) AHHQKLVFFAED (SEQ ID NO. 8), or A-Aβ(26-16) KSGVDEAFFVLK (SEQ ID NO. 6.2)) and the radionuclide conjugates of Lu-177 with the combined spacing moieties and targeting agents (e.g., S_{M}-Aβ(16-23) KHHGKLVFFAED (SEQ ID NO. 12) and AHHGKLVFFAED (SEQ ID NO. 13), S_{M}-Aβ(23-16) KHHGDEAFFVLK (SEQ ID NO. 14) and AHHGDEAFFVLK (SEQ ID NO. 15), S_{M}-K-Aβ(23-17) KHHGKDEAFFVL (SEQ ID NO. 16) or AHHGKDEAFFVL (SEQ ID NO. 17), and S_{M}-A-Aβ(23-17) KHHGADEAFFVL (SEQ ID NO. 18) or AHHGADEAFFVL (SEQ ID NO. 19)) can cross the intact or damaged blood brain barrier bind to RAGE receptors on pro-inflammatory microglia to induce apoptosis in the pro-inflammatory microglia via conversion electrons, thereby reducing the deposition of Aβ, and suppressing the propagation of hyper-phosphorylated tau protein in the brain. Through this mechanism, the presently described radionuclide conjugates can treat neurodegenerative diseases associated with pro-inflammatory microglia activity such as AD, PD, and ALS. In a preferred embodiment, the presently described radionuclide conjugates are administered to patients to treat AD. Furthermore, gamma emissions from the radionuclides (such as Sn-117m and Lu-177) can be imaged using a standard gamma camera or SPECT to assist with diagnosing conditions related to pro-inflammatory microglia, which may also be referred to herein as hyperactive microglia. In a preferred embodiment, the presently described radionuclide conjugates are administered to patients to image hyperactive microglia and diagnose, or support the diagnosis of, neurodegenerative diseases and, in particular, AD.

The structure of an exemplary radiopharmaceutical compound, Sn-117m(IBD)-R is shown below. In the embodiment shown below, the chelating agent used is IBD and there is no linking moiety. Alternatively, the same structure can be understood as Sn-117m(ABD)-L_{M}-R, wherein the chelating agent is ABD and the linking moiety comprises isothiocyanate group. In one embodiment, the R group is only a targeting agent (T_{A}) as described above. In another embodiment, the R group is a spacing moiety conjugated to a targeting agent (S_{M}-T_{A}) as described above.

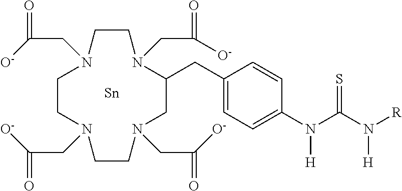

The structure of a variant of the exemplary radiopharmaceutical compound, Sn-117m(IBD)-R, is shown below. In the embodiment shown below, the chelating agent is IBD. The linker moiety shown below is an example of click chemistry (explained further below) and spans the portion between the isothiocyanate group and the R group. In one embodiment, the R group is replaced with only a targeting agent (T_{A}). In another embodiment, the R group is replaced with a spacing moiety conjugated to a targeting agent (S_{M}-T_{A}).

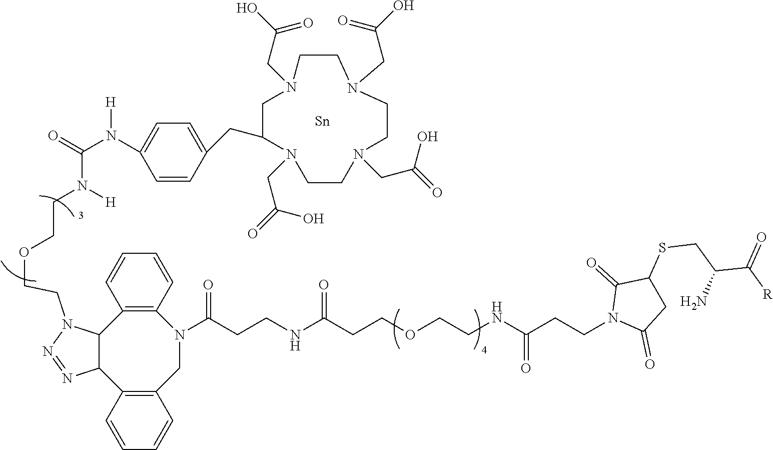

The above embodiment may comprise the use of a click chemistry process to form the linking moiety and conjugate the chelated radionuclide to the R group. In such an embodiment, the first click chemistry portion has an azide that interacts with the alkyne bond in the second click chemistry portion (in the octyne ring in the DIBO) to form a five-membered ring. The first click chemistry portion is shown to the right of the isothiocyanate portion shown below:

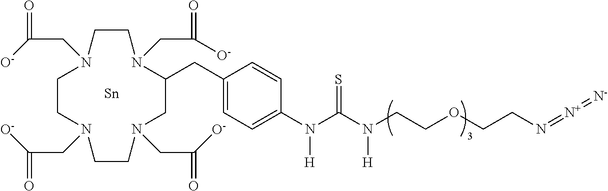

With further respect to the same embodiment, the second click chemistry portion is shown to the left of the R group below:

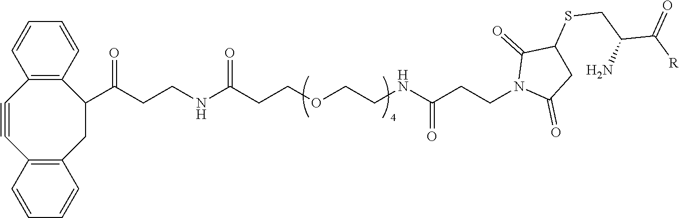

Embodiments of the radionuclide conjugate can be constructed and labeled with Sn-117m as follows, (1) Sn-117m first is attached to a bifunctional chelator such as aminobenzyl DOTA (ABD), (2) the amine group on ABD is converted to an isothiocyanate group, thereby converting the ABD to isothiocyanate DOTA (IBD), and (3) this IBD is then conjugated to a terminal lysine or alanine on the T_{M} or S_{M}-T_{M} to form Sn-117m(IBD)-R, where R is a TM or SM-TM. For example, this method may be followed for the formation of Sn-117m(IBD)-K-Aβ(23-17) also as shown above.

Embodiments of the radionuclide conjugate can be constructed and labeled with Sn-117m using Click chemistry as shown below. For this process, a terminal cysteine (Cys, C) is optionally added to the amino acid sequence of the targeting agent or combined spacing moiety and targeting agent. In the example below, the targeting agent is Aβ(16-23) KLVFFAED (SEQ ID NO. 1), which is terminally modified with cystine to form CKLVFFAED (SEQ ID NO. 20) or C-Aβ(16-23), which is subsequently coupled to DBCO-linked maleimide. However, it should be understood that in other embodiments, it is not necessary to terminally modify the targeting agents or conjugated spacing moieties and targeting agents with a cystine. Returning to the example below, the radionuclide Sn-117m is chelated to the chelating agent, which is linked to the amino acid sequence at the functional groups via the click reaction, as shown below to result in Sn-117m(IBD)-L_{M}-C-Aβ(16-23), as shown below. Similar reactions may used on other 8-amino acid targeting agents, 12 amino acid targeting agents, and/or conjugated spacing moieties and targeting agents disclosed above, with or without adding a terminal cystine.

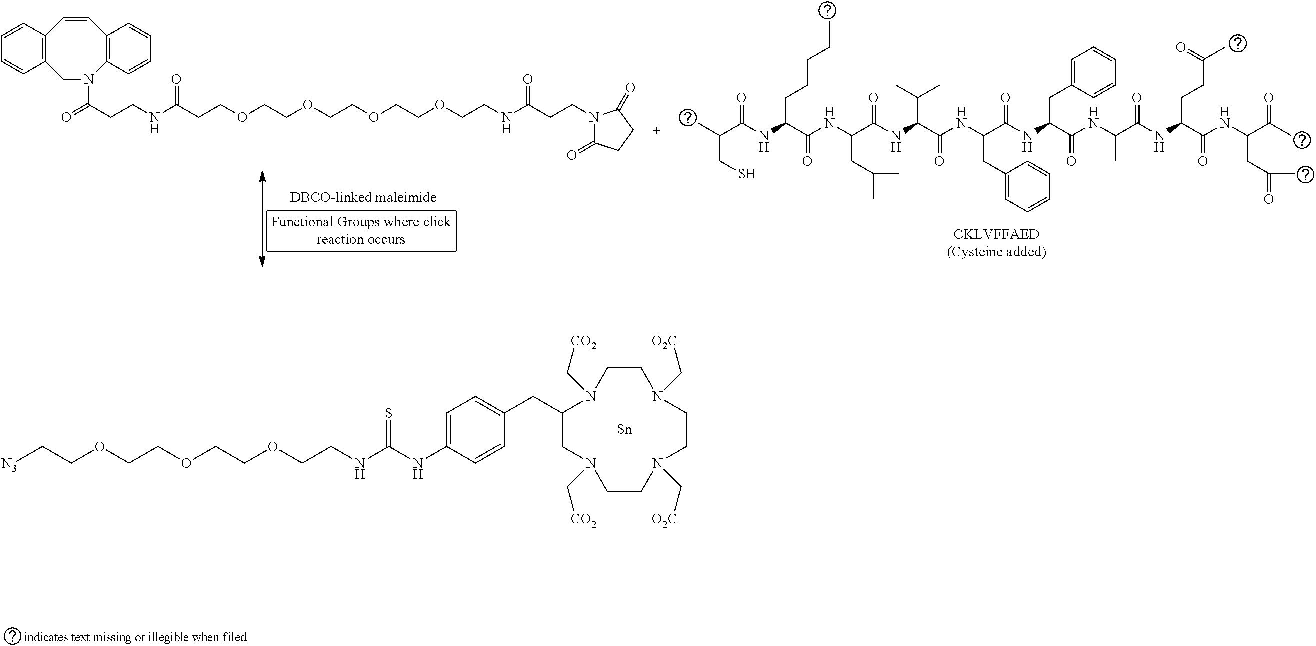

Alternate compounds may be made using click chemistry using the similar methods outlined above, with or without K, A, or C modification, using DOTA as the chelating agent instead of IBD. For example, the radiopharmaceutical composition could be synthesized using click chemistry from a Sn-117m(DOTA)-L_{P1} and a L_{P2}-R, wherein R comprises a targeting agent or a spacing moiety and targeting agent. In examples where there is an L_{P2}, it may be bound to the spacing moiety or the targeting agent as shown previously above. In one embodiment where there is a chelated radionuclide conjugated with a first linking moiety portion, L_{P1}, it is bound to the Sn-117m(DOTA) at one of the four carboxylic acid portions as shown below:

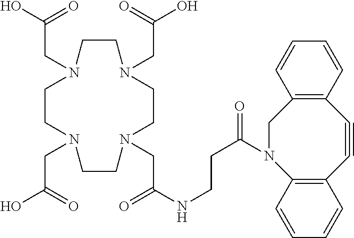 [SMILES: O=C(O)CN1CCN(CC(=O)O)CCN(CC(=O)NCCC(=O)N2Cc3ccccc3C#Cc3ccccc32)CCN(CC(=O)O)CC1]

In another embodiment, the compound below is another embodiment of the Sn-117m(DOTA)-L_{P1}:

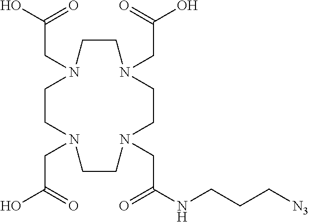 [SMILES: [N-]=[N+]=NCCCNC(=O)CN1CCN(CC(=O)O)CCN(CC(=O)O)CCN(CC(=O)O)CC1]

For either of the example molecules shown above for the Sn-117m(DOTA)-L_{P1}, in one embodiment the following may comprise the L_{P2}-R:

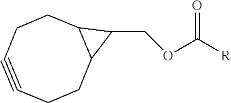

For either of the example molecules shown above for the Sn-117m(DOTA)-L_{P1}, in one embodiment the following may comprise the L_{P2}-R:

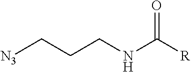

Embodiments of the radionuclide conjugate are capable of crossing the blood brain barrier into the CNS to target hyperactive (‘pro-inflammatory’) microglia when given peripherally. In embodiments, the radionuclide conjugate is administered intravenously. In other embodiments, radionuclide conjugate is delivered intra-arterially, such as into the carotid artery, which allows for the use of higher concentrations (i.e., higher localizing dosage) and in lower volume when administered, as compared to the dose needed when administered intravenously.

In embodiments of the invention, the radionuclide conjugate is administered in an amount effective to image pro-inflammatory microglia, induce apoptosis in pro-inflammatory microglia, or both image and induce apoptosis in pro-inflammatory microglia, which can in turn, treat a neurodegenerative disease such as AD, PD, and ALS. In a preferred embodiment, the radionuclide conjugate is injected at a dose sufficient to treat AD.

The amount effective to treat a neurodegenerative disease caused, at least in part, by pro-inflammatory microglia, is an amount that delivers a sufficient dose of the radionuclide conjugate to the central nervous system to result in a hormetic response in the central nervous system. The hormetic response may include inducing apoptosis in pro-inflammatory microglia, without inducing wider spread necrosis of tissue in the central nervous system. In embodiments, the amount administered is effective to image pro-inflammatory microglia in the subject. In further embodiments, the amount administered is effective to both induce apoptosis and image pro-inflammatory microglia in the subject. In embodiments, imaging can be conducted with a gamma camera or with single-photon emission computerized tomography (“SPECT”). In embodiments of the invention, the effective dose is below a dose that result in DNA, RNA, and polymerase fracturing. The amount administered can vary depending on the severity of the neurodegenerative disease, the route of administration, and the specific activity of the radionuclide. In an embodiment, the amount administered is sufficient to deliver a dose in a range from 100 μCi to 100 mCi to the central nervous system. In another embodiment, the amount administered is sufficient to deliver to the central nervous system a dose in a range from 500 μCi to 10 mCi, or a dose in a range from 3 mCi to 100 mCi. Lower doses, such as from 500 μCi to 10 mCi, may be administered via arterial injection, such as into the carotid artery. Higher dosages, such as 3 mCi to 100 mCi may be administered via intravenous injection.

### EXAMPLES

### Example 1

Animal studies—Truncated portions of the Aβ peptide amino acid chain have been identified as critical in binding to RAGE. These 8 amino acid chains have demonstrated critical binding affinity. Attaching Sn-117m to these amino acids allows for targeting pro-inflammatory microglia within the radioisotope for subsequent therapeutic action.

Four APPSWE Model 2789 mice (AD) and 4 C573L/6 mice (normal) receive an injection of 50 μCi of Sn-IBD-Aβ(16-23) per tail vein at day 0.

Additionally, four APPSWE Model 2789 mice (AD) and 4 C57B L/6 mice (normal) receive an injection of 50 μCi of Sn-IBD-K-Aβ(23-17) per tail vein at day 0.

All the animals are sacrificed on day 3 post Sn-Aβ-AA injection and brains preserved and autoradiography (AR) binding localization in AD-specific areas mapped.

Findings—Histopathological comparison and autoradiography dosimetry are measured in each mouse brain in comparative anatomic regions. Normal mouse brain AR distribution of RAGE binding should be minimal in cortical and hippocampal areas whereas the RAGE expressing mice will show an increase in RAGE binding with both the Sn-IBD-Aβ(16-23) and the Sn-IBD-K-Aβ(23-17).

### Example 2

Four APPSWE Model 2789 mice (AD) and 4 C57BL/6 mice (normal) receive an injection of 50 μCi of Sn-IBD-Aβ(16-23) or 50 μCi of Sn-IBD-K-Aβ(23-17) per tail vein at day 0. The animals are sacrificed on day 3 post injection and brains preserved and autoradiography (AR) binding localization in AD-specific areas mapped. A comparison of binding localization between the two molecules is performed to verify binding of the radionuclide conjugate in the brains of the AD mice.

Systemically injected target RAGE expressed on the plasma membrane of pro-inflammatory microglia, irradiating them with conversion electrons, disrupting the neuro-inflammatory cascade that is implicated in several neurological disorders. The resulting radionuclide conjugate is capable of binding to the cellular RAGE that results in inhibition of pro-inflammatory microglia and is believed to occur without the internal cellular triggering of the cytoplasmic tail of RAGE and the intracellular effector, diaphanous-1.

In embodiments of the invention, the irradiation of microglia may be conducted using other radioisotopes including beta energy emitters to the cellular RAGE to result in inhibition of pro-inflammatory microglia and occurs without the internal cellular triggering of the cytoplasmic tail of RAGE and the intracellular effector, diaphanous-1.

### Example 3

As used in the context of Example 3, Sn(II) and Sn(IV) are understood to refer to the respective oxidation states of Sn-117m. A peptide containing 12 amino acids containing a DOTA chelating group was combined with an aqueous solution of Sn-117m(II) using a 1:1 metal to chelant ratio and the solution was allowed to stand at room temperature for 30 minutes. A small amounts of hydrogen peroxide was added to convert Sn(II) to Sn(IV). Reverse phase high performance liquid chromatography (HPLC) was used to analyze the resulting solution. Two peaks with a similar retention time as the non-metalated peptide resulted consistent with the formation of two Sn(IV)-DOTA-peptide species. These results show that it is possible to prepare Sn chelates with DOTA based conjugates if the starting Sn oxidation state is +2.

### Example 4

As used in the context of Example 4, Sn(II) and Sn(IV) are understood to refer to the respective oxidation states of Sn-117m. An aqueous solution of the peptide-DOTA conjugate of example 1 was combined with an aqueous solution (100 μCi) of Sn-117m in the +4-oxidation state. The pH was adjusted to 2 with HCl and the solution was heated at 100 C for two hours. Analysis by reverse phase HPLC of the solution after heating showed no radioactive peaks suggesting that Sn (IV) did not form chelates with the peptide-DOTA conjugate. The lack of any radioactive peak is consistent with Sn-117m forming an insoluble hydroxide species that does not make it through the HPLC column. The results show that it was not possible to form a Sn chelate even using harsh conditions when starting with Sn in the +4-oxidation state.

### Example 5

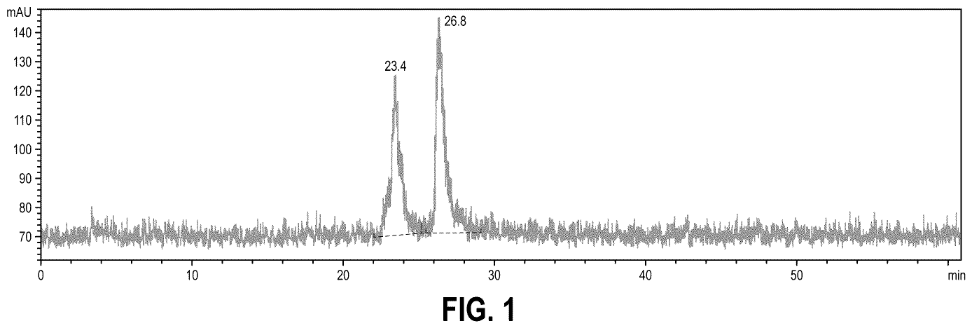

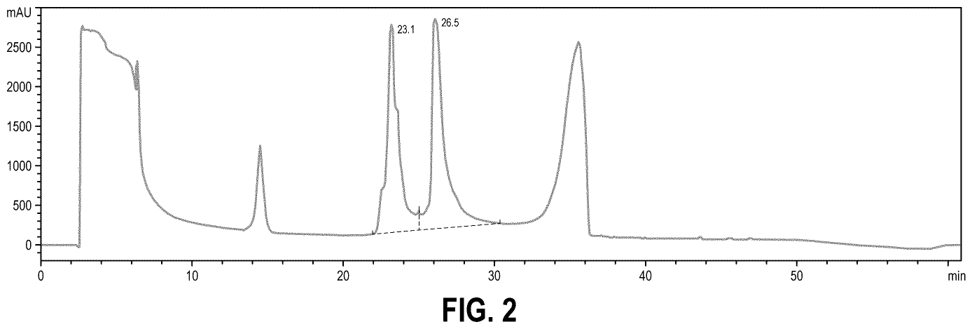

As used in the context of Example 5, Sn(II) and Sn(IV) are understood to refer to the respective oxidation states of Sn-117m. A mass of 0.5 mg of the peptide-DOTA conjugate of example 1 was dissolved in 50 μL of DMSO. An activity of 302 μCi of Sn-117m as the tetraiodide was dissolved with 50 μL of DMSO. The specific activity of the Sn-117m was low such that moles of Sn were about equal to the moles of DOTA-peptide. The solutions were combined into the radioactive vial and about 2 mg of solid anhydrous potassium carbonate was added. The solution was heated at 60° C. overnight then analyzed by reverse phase HPLC. Two radioactive peaks with retention times of 23 and 26 minutes were observed in the UV spectrum with corresponding radioactive peaks. The retention time of the unchelated DOTA-peptide was 23.8 minutes. The slight shift in retention time from the unchelated to the two radioactive peaks is evidence of complex formation resulting in two conformations of the Sn-DOTA-peptide. There were no other radioactive peaks consistent with a high yield reaction. FIG. 1 shows the chromatographs of the Sn-117m(DOTA)-peptide chelate. FIGS. 1 and 2 each show one chromatograph with both a UV and a radioactive detector in series. FIG. 1 shows the HPLC data wherein only those compounds that exhibit radioactivity give peaks. FIG. 2 shows all compounds that went throughout the HPLC. Accordingly, it is clear from the two peaks shown in the FIG. 1 of the HPLC data showing radioactivity that only the chelating agents were radioactive, meaning that the radioisotope was completely chelated and is easily separated from the other components in solution. Without being bound by theory, it is believed that the retention times of the radioactive peaks are slightly larger than those of the UV peaks because the detectors are in series with the radiation detectors after the UV detectors.

### Examples 6-10

The following Examples 6-10 are related to DOTA-Polypeptide-Lu chelation and click reaction, and were particularly used to evaluate and optimize the stepwise development of a DOTA-DBCO-Polypeptide conjugate and subsequent Lu-177 chelation under varying conditions of pH, temperature and molar ratios. Characterization was by HPLC and thin layer chromatography (TLC). Materials and stock solutions used are shown in Table 1, below. And the general reaction to form the conjugate is shown in FIG. 12, with FIG. 13 showing the two stereoisomer products.

TABLE 1
Material and Stock Solutions

| | Stock | | | | |
|---|---|---|---|---|---|
| Reagent | Concentration | | Matrix | Storage | Notes |
| DOTA-DBCO | 0.9 | mg/mL | DI water | RT | MW 777 |
| Polypeptide-Azide | 0.9 | mg/mL | DI water | RT | MW 1600 |
| Lu-177 Chloride | 7.44 | mCi/mL | 0.04M HCL | RT | — |
| Stock | | | | | |
| Ammonium acetate | 0.5M | | — | 4-8 C | — |
| buffer, pH 6.5 | | | | | |
| Sodium Citrate | 0.1M | | — | RT | — |
| buffer pH 5 | | | | | |
| DI water | 18 | Ω | — | — | Used for |

dilutions

### Example 6. DOTA-DBCO Characterization by HPLC

The objective of this example was to evaluate purity and retention of DOTA-DBCO standard under analytical HPLC conditions. The particular HPLC method and conditions used in this example are shown in Table 2, below.

TABLE 2
HPLC method description
HPLC method

| Method | BED_LC4.M | |
|---|---|---|
| Column: | C18, 250 × 4.6 mm, 5 μm | |
| Flow rate | 1.0 mL/min | |
| Pressure limits | 0-300 mbar | |
| Run time | 16 min | |
| Post-run time | 4.0 min | |
| Mobile phase | A: Water + 0.1% TFA | |
| | B: Acetonitrile + 0.1% | |
| | [symbol] | |
| Gradient timetable | Time (min) | % B |
| | 0.00 | 0 |
| | 3.00 | 0 |
| | 10.00 | 38 |
| | 10.10 | 50 |
| | 12.00 | 50 |
| | 12.10 | 0 |
| | 16.00 | 0 |

| | |
|---|---|
| Detector | UV at 220 nm |
| Injection volume | 10 μL |

[symbol]

indicates data missing or illegible when filed

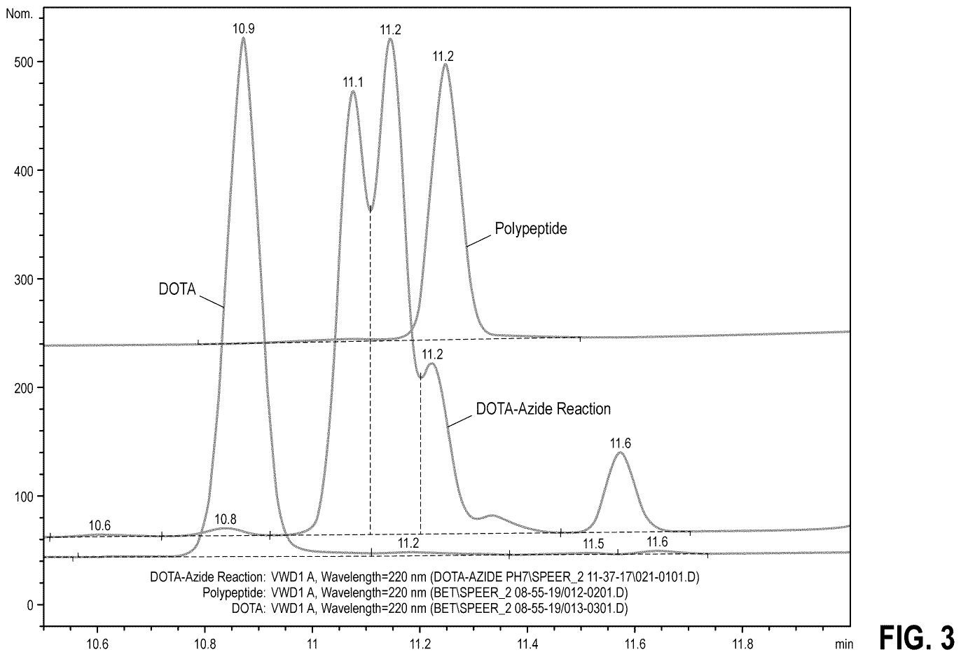

As can be seen in FIG. 3, a single, sharp peak was observed at Rt=10.87 min, with no significant secondary peaks. The retention was consistent across duplicate injections, confirming method reproducibility.

### Example 7. 12 Amino Acid Polypeptide Characterization by HPLC

The objective of this example was to characterize the 11-amino acid polypeptide-azide by HPLC. The HPLC method used in this example is the same BED LC4.M method used above with respect to Example 6.

With reference to FIG. 3, a sharp, symmetrical peak appears at Rt=11.24 min, with high signal intensity. No additional significant peaks were detected, indicating high purity and good stability under analytical conditions. These results were consistent across multiple injections.

### Example 8. Conjugation (Click Reaction) of DOTA-DBCO+Polypeptide-Azide

The objective of this example was to confirm successful conjugation between DOTA-DBCO and the 11-amino-acide-polypeptide-azide under mild aqueous conditions.

Reaction conditions: Table 3 below shows rection conditions for this example. The reaction was prepared under equimolar conditions. The ratio of the stock solution was calculated based on the concentration of each stock material (0.9 mg/mL). The ratio used was 1:2.07 (DOTA: Polypeptide-Azide). The pH was adjusted to 7.0 using HEPES as a buffer. The reaction was incubated at room temperature for 30 minutes.

TABLE 3
Reaction conditions

| Parameter | Value |
|---|---|
| Ratio | 1:2 (DOTA:Polypeptide) |
| pH | 7.0 |
| Temperature | RT (~22° C.) |
| Incubation | 30 min |
| Buffer | HEPES |
| Analysis | BED_LC4.M HPLC |

Results: Referring to FIG. 3, a new doublet appeared between 11.07-11.28 min, distinct from DOTA (10.87 min) and polypeptide (11.24 min). The shift in retention confirms formation of new chemical entity—the DOTA-Polypeptide conjugate. The click reaction was replicated using the same reaction ratio (1:2, DOTA: Polypeptide, 0.38 mM each). All trials produced identical chromatographic profiles, confirming reproducibility of the conjugation step.

### Example 9. Chelation Optimization of DOTA with ⁷⁷-Lu

The objective of this example was to optimize chelation between DOTA-DBCO and Lu-177 chloride by studying the effects of pH, temperature, and DOTA:Lu ratio. The goal was to identify the best parameters for efficient metal chelation before proceeding to the conjugation with the polypeptide.

Reaction conditions: Chelation trials were performed using DOTA-DBCO and Lu-177 chloride to evaluate the effect of pH and incubation time on complex formation. Reactions were prepared in 0.5 M ammonium acetate buffer adjusted to pH 5.5, 6.0, 6.5 and 7.0. with each mixture incubated for two hours. TLC analyses were conducted in parallel using 0.9% saline and 0.1 M sodium citrate mobile phases. A Lu-177 control was analyzed in parallel to confirm the migration pattern of free lutetium. Reaction parameters are shown in Table 4, below.

TABLE 4
Reaction parameters

| DOTA-DBCO | | | Lu-177 Chloride | | |
|---|---|---|---|---|---|
| Stock Concentration | 0.9 | mg/mL | Activity Concentration | 7.44 | mCi/mL |

| | | | | |
|---|---|---|---|---|
| MW | 777 | Activity for reaction | 200 | uCi |

| Molarity | 1.158 mM-11.58 μM | | Dilution factor | 1:100, 10 μL Lu-177 + |
|---|---|---|---|---|
| Dilution factor | 1:100, 10 μL DOTA + | | | 990 μL 0.04N HCL |
| | 990 μL DI water | | | |
| Working molarity | 11.5 | μM | Working dilution | 7.25 uCi/μL |

activity

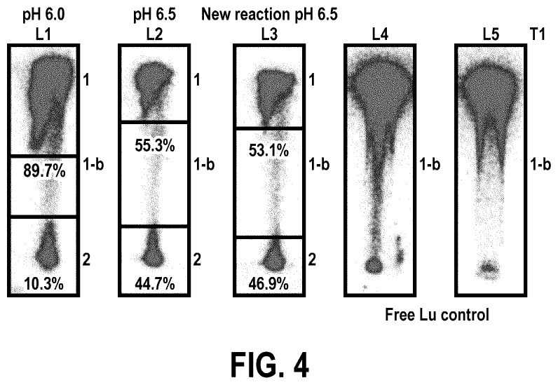

TLC results (with reference to FIG. 4): In saline, radioactivity remained at the origin, confirming the presence of unchelated Lu-177; in contrast, the citrate mobile phase enabled migration of the chelated complex, allowing qualitative estimation of yield. Chelation efficiency increased progressively with pH up to 6.5, but decreased again at pH 7.0, suggesting that the optimal pH range for Lu-177 chelation is between 6.0 and 6.5.

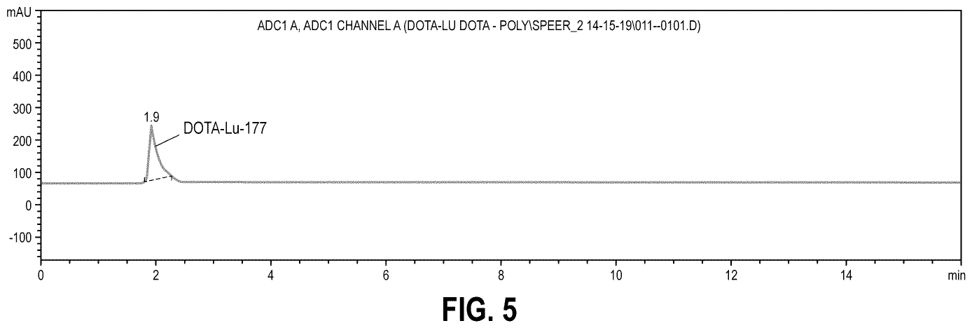

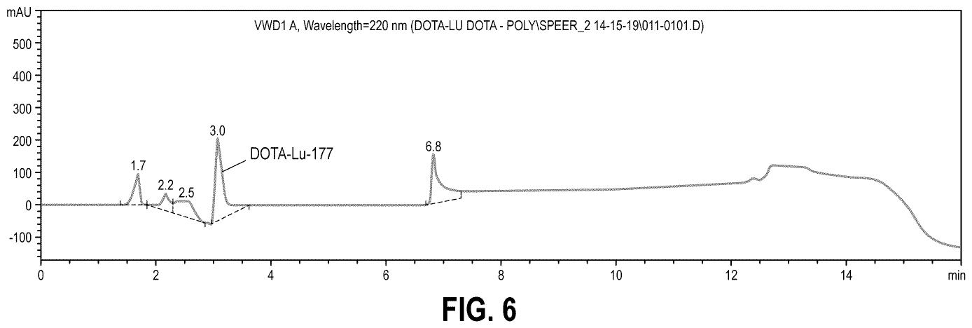

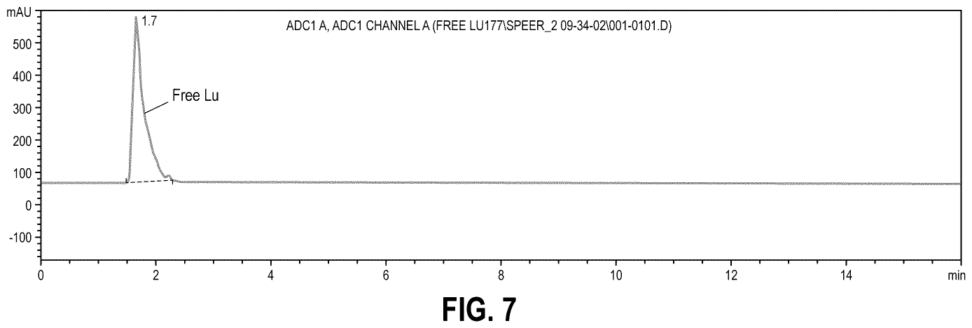

HPLC results (with reference to FIGS. 5, 6, and 7): To verify chelation efficiency, HPLC was performed on the DOTA-Lu-177 reaction mixture at pH 6.5 alongside a free Lu-177 control. The free Lu-177 control showed an early peak at RT≈1.6 min, corresponding to unchelated lutetium (see FIG. 7). In contrast, the DOTA-Lu-177 chromatogram did not exhibit the early signal (RT≈1.6 min) and the appearance of a well-defined product peak at RT≈6.8 min, confirmed successful chelation of Lu-177 to the DOTA ligand. No residual Lu peaks were detected at the void volume. (See FIGS. 5 and 6.)

### Example 10. Click Reaction of DOTA-⁷⁷-Lu-DBCO with Polypeptide-Azide

The objective of this example was to perform the click coupling of the pre-chelated DOTA-Lu-DBCO complex with the 11-amino-acid polypeptide-azide at optimized pH 6.5 and verify conjugation by HPLC and TLC.

Reaction: The pre-chelated DOTA-Lu-DBCO complex obtained at pH 6.5 (46.9%) was used as the starting material for the click conjugation step. A total 100 μL of this chelated fraction was combined with 4.1 μL of the polypeptide-azide stock solution (0.9 mg/mL≈0.5625 mM). The reaction mixture was then adjusted to pH 6.5 with 0.5 M ammonium acetate buffer to maintain the optimal conditions previously determined for chelation. The solution was incubated at 37° C. for 2 hours under gentle shaking. Following incubation, the reaction was analyzed by HPLC using dual UV (220 nm) and radiometric detection to confirm formation of the conjugated product and by TLC in 0.1 M sodium citrate to assess radiochemical purity and verify the absence of free Lu-177. Reaction parameters are shown in Table 5.

TABLE 5
Reaction parameters

| Chelated precursor | | Polypeptide-azide Stock | | |
|---|---|---|---|---|
| DOTA-Lu-DBCO, pH 6.5 | | Stock concentration | 0.9 | mg/mL |
| chelation product, 46.9% | | Working molarity | 0.56 | mM |
| Volume | 100 μL | Volume | 4.1 | μL |

Conditions

| | |
|---|---|
| pH | 0.5M Ammonium acetate for pH adjustment at 6.5 |
| Incubation | 2 hours at 37° C. with constant gentle shaking |
| conditions | |
| HPLC method | BED_LC4.M |
| TLC mobile phase | 0.1M sodium citrate |

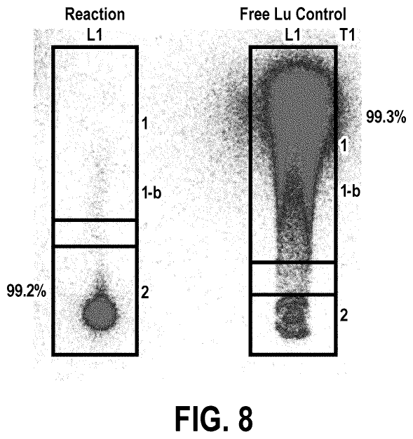

TLC results (with reference to FIG. 8): TLC developed in 0.1 M sodium citrate confirmed the chelated and conjugated nature of the product. The product remained at the origin, while the free Lu control migrated with the solvent front. This is consistent with the expected behavior of a stable DOTA-Lu complex. No intermediate migration zones were observed, indicating a fully chelated complex.

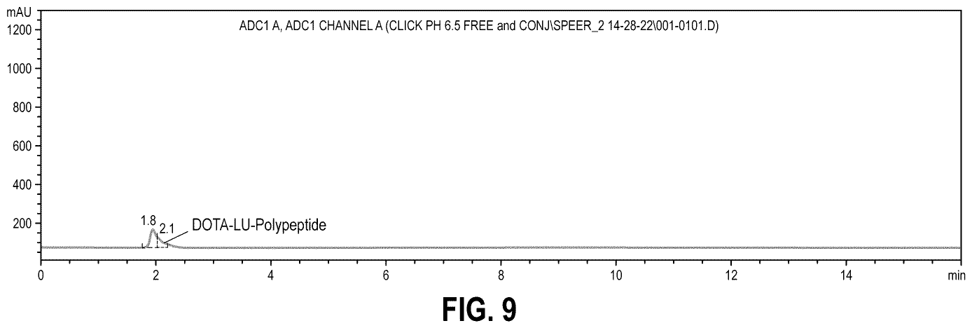

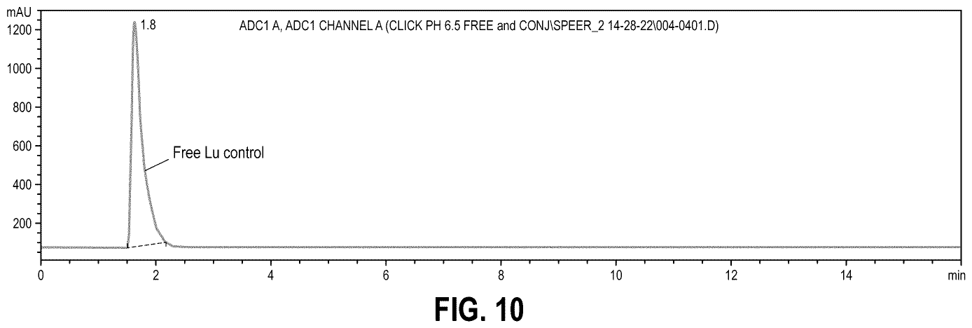

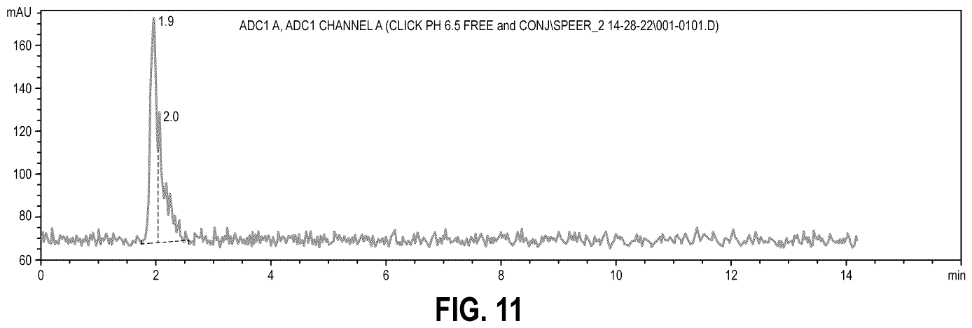

HPLC results (with reference to FIGS. 9, 10, and 11): Radiometric chromatogram showed a difference in retention in peak profile between the free Lu-177 control and the conjugate. The free Lu-177 control exhibited a sharp, narrow peak at Rt≈1.7 min, characteristic of unchelated lutetium chloride (see FIG. 10). In contrast, and referring to FIGS. 9 and 11) the chelate showed a broader, shifted peak at Rt≈1.98 min, indicating a heavier, more polar complex consistent with a chelated species. The absence of any additional earlier radioactive peaks in the conjugated samples suggests that all Lu-177 was incorporated into the final DOTA-Lu-Polypeptide structure, with no detectable free Lu-177.

While specific embodiments have been described in considerable detail to illustrate the present invention, the description is not intended to restrict or in any way limit the scope of the appended claims to such detail. The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope of the general inventive concept.

What is claimed is:

1. A radiopharmaceutical compound configured to induce apoptosis, image, or both induce apoptosis and image pro-inflammatory microglia in the central nervous system of a subject, the radiopharmaceutical compound comprising:
a targeting agent, T_{A}, wherein the targeting agent is configured to bind with the RAGE receptor;
a chelated radionuclide, Rad(C), comprising a chelating agent, C, and a radionuclide, Rad, wherein the radionuclide is included in an effective amount to induce apoptosis, image, or both induce apoptosis and image pro-inflammatory microglia; and
a spacing moiety, S_{M}, wherein the spacing moiety is conjugated with the targeting agent, and wherein the spacing moiety reduces steric hinderance for the radiopharmaceutical compound when compared to otherwise identical radiopharmaceutical compounds not including the spacing moiety, and wherein the inclusion of spacing moiety results in a water soluble radiopharmaceutical compound, and wherein an otherwise identical radiopharmaceutical compound not including the spacing moiety would not be water soluble.

2. The radiopharmaceutical compound of claim 1, wherein the radionuclide is Sn-117m.

3. The radiopharmaceutical compound of claim 1, wherein the chelating agent is selected from the group consisting of diethylene triamine pentaacetic acid (DTPA), 1,4,7,10-Tetraazacyclododecane-1,4,7,10-tetraacetic acid (DOTA), aminobenzyl DOTA (ABD), and isothiocyanatebenzyl DOTA (IBD).

4. The radiopharmaceutical compound of claim 1, wherein the targeting agent comprises a truncated portion of the amyloid β (Aβ) peptide amino acid chain having at least the 7 amino acid length between the seventeenth and twenty-third amino acid, wherein the truncated portion is configured to be positioned distally from the radionuclide; or wherein the targeting agent is an amino acid chain selected from the group consisting of KLVFFAED (SEQ ID NO 1), KDEAFFVL (SEQ ID NO 2), and ADEAFFVL (SEQ ID NO 3).

5. The radiopharmaceutical compound of claim 1, wherein the spacing moiety is an amino acid chain having a length greater than or equal to 2 amino acids and less than or equal to 6 amino acids; or
wherein the spacing moiety is selected from the group consisting of KHHG (SEQ ID NO 10) and AHHG (SEQ ID NO 11), and wherein the terminal lysine (K) or terminal alanine (A) of the spacing moiety is configured to conjugate with either the chelated radionuclide or a linking moiety.

6. The radiopharmaceutical compound of claim 5, wherein the spacing moiety is KHHG (SEQ ID NO 10), and wherein the spacing moiety and targeting agent are conjugated to form a combined R group selected from the list consisting of KHHGKLVFFAED (SEQ ID NO 12), KHHGDEAFFVLK (SEQ ID NO 14), KHHGKDEAFFVL (SEQ ID NO 16), and KHHGADEAFFVL (SEQ ID NO 18); or
wherein the spacing moiety is AHHG (SEQ ID NO 10), and wherein the spacing moiety and targeting agent are conjugated to form a combined R group selected from the list consisting of AHHGKLVFFAED (SEQ ID NO 13), AHHGDEAFFVLK (SEQ ID NO 15), AHHGKDEAFFVL (SEQ ID NO 17), and AHHGADEAFFVL (SEQ ID NO 19).

7. The radiopharmaceutical compound of claim 1, wherein the chelated radionuclide is conjugated with the spacing moiety.

8. The radiopharmaceutical compound of claim 1 further comprising a linking moiety, L_{M}, wherein the linking moiety is conjugated with the chelated radionuclide and the spacing moiety.

9. The radiopharmaceutical compound of claim 8, wherein the linking moiety comprises a first linking moiety portion and a second linking moiety portion, and wherein the first linking moiety portion and the second linking moiety portion are configured to be joined using a click chemistry reaction.

10. The radiopharmaceutical compound of claim 9, wherein the first linking moiety portion and the second linking moiety are configured to be joined by a click chemistry reaction between a terminal azide and a cyclooctyne.

11. The radiopharmaceutical compound of claim 10, wherein the portion of the first or second linking moiety portion containing the terminal azide is selected from a list consisting of azido-triethylene glycol-NHS ester, azido-tetraethylene glycol-NHS ester, azido-pentaethylene glycol-NHS ester, azido-triethylene glycol-maleimide, azido-tetraethylene glycol-maleimide, azido-pentaethylene glycol-maleimide, azido-triethylene glycol-amine, azido-tetraethylene glycol-amine, and azido-pentaethylene glycol-amine.

12. The radiopharmaceutical compound of claim 10, wherein the portion of the first or second linking moiety portion containing the cyclooctyne is selected from a list consisting of a difluorooctyne (DIFO), a dibenzylcyclooctyne (DIBO), an azadibenzylcyclooctyne (ADIBO), and cylcopropanecyclooctyne (CPO).

13. The radiopharmaceutical compound of claim 1, wherein the radionuclide is chelated in a non-aqueous process.

14. The radiopharmaceutical compound of claim 1, wherein the radionuclide has a specific activity selected from the group consisting of (1) greater than or equal to 100 Ci/g and less than or equal to 25,000 Ci/g, and (2) greater than or equal to 100 Ci/g and less than or equal to 1,000 Ci/g.

15. The radiopharmaceutical compound of claim 1, wherein the radiopharmaceutical compound is configured to induce apoptosis in pro-inflammatory microglia.

16. A method of inducing apoptosis in pro-inflammatory microglia comprising administering the radiopharmaceutical compound of claim 1 to a subject in an effective amount.

17. The method of claim 16, wherein the radiopharmaceutical compound is administered to the subject via systemic injection to the subject.

18. The method of claim 17, wherein the radiopharmaceutical compound is injected in a manner selected from the group consisting of intra arterially to the subject, injected into the carotid artery, and injected intravenously into the subject.

19. The method of claim 16, wherein the amount of the radiopharmaceutical compound administered to the subject is sufficient to deliver a dose to the central nervous system in a range selected from the group consisting of 100 μCi to 100 mCi, 500 μCi to 10 mCi, and 3 mCi to 100 mCi.

20. A method of synthesizing a radiopharmaceutical compound configured to induce apoptosis, image, or both induce apoptosis and image pro-inflammatory microglia in the central nervous system of a subject, the method comprising:
chelating a radionuclide, Rad, with a chelating agent, C, to form a chelated radionuclide, Rad(C), wherein chelating comprises:
dissolving a radionuclide and a chelating agent together in a solvent under mild conditions to form a dissolved mixture;
conjugating the chelating agent or chelated radionuclide with a substituent including a targeting agent, T_{A}.

21. The method of claim 20, wherein the chelated radionuclide is conjugated with a substituent including a targeting agent after chelating the radionuclide, or wherein the chelating agent is conjugated with a substituent including a targeting agent prior to chelating the radionuclide, wherein the order of steps enables a process wherein the substituent including a targeting agent is not subjected to harsh conditions.

22. The method of claim 20, wherein the radionuclide is Sn-117m.

23. The method of claim 20, wherein the chelating agent is selected from a list consisting of diethylene triamine pentaacetic acid (DTPA), 1,4,7,10-Tetraazacyclododecane-1,4,7,10-tetraacetic acid (DOTA), aminobenzyl DOTA (ABD), and isothiocyanatebenzyl DOTA (IBD).

24. The method of claim 20, wherein the targeting agent comprises a truncated portion of the amyloid β (Aβ) peptide amino acid chain having at least the 7 amino acid length between the seventeenth and twenty-third amino acid, wherein the truncated portion is configured to be positioned distally from the radionuclide, or
wherein the targeting agent is an amino acid chain selected from the group consisting of KLVFFAED (SEQ ID NO 1), KDEAFFVL (SEQ ID NO 2), ADEAFFVL (SEQ ID NO 3), KHHQKLVFFAED (SEQ ID NO 6), KSGVDEAFFVLK (SEQ ID NO 7), AHHQKLVFFAED (SEQ ID NO 8), and ASGVDEAFFVLK (SEQ ID NO 9).

25. The method of claim 20, wherein conjugating the chelating agent or chelated radionuclide with a substituent including a targeting agent comprises conjugating the targeting agent with the chelating agent or the chelated radionuclide.

26. The method of claim 20, further comprising synthesizing the substituent including a targeting agent, wherein synthesizing the substituent including a targeting agent comprises conjugating the targeting agent with a spacing moiety, S_{M}.

27. The method of claim 26, wherein the spacing moiety is selected from the list consisting of KHHG (SEQ ID NO 10) and AHHG (SEQ ID NO 11).

28. The method of claim 26, wherein conjugating the chelating agent or chelated radionuclide with a substituent including a targeting agent comprises conjugating the spacing moiety with the chelating agent or the chelated radionuclide.

29. The method of claim 26, wherein synthesizing the substituent including a targeting agent further comprises conjugating the spacing moiety with a linking moiety, and wherein conjugating the chelating agent or chelated radionuclide with a substituent including a targeting agent comprises conjugating the linking moiety with the chelating agent or the chelated radionuclide.

30. The method of claim 26, wherein synthesizing the substituent including a targeting agent further comprises conjugating the spacing moiety with a first click chemistry portion, and wherein conjugating the chelating agent or chelated radionuclide with a substituent including a targeting agent comprises:
conjugating a second click chemistry portion with the chelating agent or the chelated radionuclide; and
conjugating the first click chemistry portion and the second click chemistry portion to form the radiopharmaceutical compound.

31. The method of claim 30, wherein the first click chemistry portion and the second click chemistry portion are joined together via a click chemistry reaction between a terminal azide and a cyclooctyne.

32. The method of claim 31, wherein the terminal azide is selected from a list consisting of azido-triethylene glycol-NHS ester, azido-tetraethylene glycol-NHS ester, azido-pentaethylene glycol-NHS ester, azido-triethylene glycol-maleimide, azido-tetraethylene glycol-maleimide, azido-pentaethylene glycol-maleimide, azido-triethylene glycol-amine, azido-tetraethylene glycol-amine, and azido-pentaethylene glycol-amine.

33. The method of claim 31, wherein the cyclooctyne is selected from a list consisting of a difluorooctyne (DIFO), a dibenzylcyclooctyne (DIBO), an azadibenzylcyclooctyne (ADIBO), and cylcopropanecyclooctyne (CPO).

34. The method of claim 20, wherein synthesizing the substituent including a targeting agent further comprises conjugating the targeting agent with a first click chemistry portion, and wherein conjugating the chelating agent or chelated radionuclide with a substituent including a targeting agent comprises:
conjugating a second click chemistry portion with the chelating agent or the chelated radionuclide; and
conjugating the first click chemistry portion and the second click chemistry portion to form the radiopharmaceutical compound.

35. The method of claim 34, wherein the first click chemistry portion and the second click chemistry portion are joined together via a click chemistry reaction between a terminal azide and a cyclooctyne.

36. The method of claim 35, wherein the terminal azide is selected from a list consisting of azido-triethylene glycol-NHS ester, azido-tetraethylene glycol-NHS ester, azido-pentaethylene glycol-NHS ester, azido-triethylene glycol-maleimide, azido-tetraethylene glycol-maleimide, azido-pentaethylene glycol-maleimide, azido-triethylene glycol-amine, azido-tetraethylene glycol-amine, and azido-pentaethylene glycol-amine.

37. The method of claim 35, wherein the cyclooctyne is selected from a list consisting of a difluorooctyne (DIFO), a dibenzylcyclooctyne (DIBO), an azadibenzylcyclooctyne (ADIBO), and cylcopropanecyclooctyne (CPO).

38. The method of claim 20, wherein the solvent is a nonaqueous solvent, and is selected from a list consisting of DMSO, THF and DMF.

39. The method of claim 20, wherein chelating further comprises, after dissolving the radionuclide and chelating agent, at least one step selected from the list consisting of a base treatment step, a heat treatment step, an incubation step, and a purification step.

40. The method of claim 39, wherein the base treatment step is performed and comprises adding an amount of base effective to scavenge hydrogen ions formed in the dissolved mixture.

41. The method of claim 39, wherein the heat treatment step is performed and comprises heating the dissolved mixture to a temperature greater than or equal to 20° C. and less than or equal to 60° C. for a duration greater than or equal to 1 hour.

42. The method of claim 39, wherein the incubation step is performed and comprises incubating the dissolved mixture at room temperature for a duration greater than or equal to 12 hours.

43. The method of claim 39, wherein the purification step is performed and comprises separating out the chelated radionuclide from the solvent using a separation technique selected from the list consisting of chromatographic separation, nanofiltration, ultrafiltration, and evaporation of the solvent.

44. A radiopharmaceutical compound containing a targeting moiety and an isotope wherein the targeting moiety targets amyloid-beta peptide receptors.

45. The radiopharmaceutical compound of claim 44, wherein the targeting construct contains an 8 amino acid sequence consisting of the sequence Lys-Leu-Val-Phe-Phe-Ala-Glu-Asp-OH.

46. The radiopharmaceutical compound of claim 44, wherein the isotope is selected from the group consisting of Sn-117m, Zr-89, Lu-177, Ac-225, Pb-212, In-111, Zr-89, Ga-68, Ga-67, Sm-153, Ra-223, Tb-161.

47. The radiopharmaceutical compound of claim 46, where a chelating agent is used to attach the isotope to the targeting moiety.

48. The radiopharmaceutical compound of claim 47, wherein the chelating agent is chosen from DOTA, DOTAGA, DTPA, Macropa™.

49. The radiopharmaceutical compound of claim 48, wherein the targeting moiety is separated from a radioactive portion and click chemistry is used to combine the two together.

50. The radiopharmaceutical compound of claim 49, wherein the targeting moiety comprises functional groups for water solubility and functionality to react with the radioactive portion.

51. The radiopharmaceutical compound of claim 50, wherein the targeting moiety includes Azido-PEG3-His-His-Gln-Lys-Leu-Val-Phe-Phe-Ala-Glu-Asp-OH.

52. The radiopharmaceutical compound of claim 49, wherein the chelating agent is DOTA.

53. The radiopharmaceutical compound of claim 52, wherein the radioactive portion comprises a click chemistry moiety.

54. The radiopharmaceutical compound of claim 53, wherein the radioactive portion is DO3A-DBCO, (1,4,7,10-Tetraazacyclododecane-1,4,7-tris(acetic acid)-10-[3-oxo-3-(5-azadibenzocyclootyne)acetamide].

55. The radiopharmaceutical compound of claim 51, wherein the radioactive portion comprises Sn-117m.

56. A process of forming the radiopharmaceutical compound of claim 55, wherein the Sn-117m is reacted with the DOTA containing moiety as a first step followed by the click reaction to the targeting moiety.

57. A process of diagnosing or treating a subject with Alzheimer's disease comprising administering the radiopharmaceutical compound of claim 44 to a subject with Alzheimer's disease.


SEQUENCE LISTING (20 sequences)

SEQ ID NO: 1 - protein, 8 aa, Homo sapiens
KLVFFAED

SEQ ID NO: 2 - protein, 8 aa, synthetic construct
KDEAFFVL

SEQ ID NO: 3 - protein, 8 aa, synthetic construct
ADEAFFVL

SEQ ID NO: 4 - protein, 11 aa, Homo sapiens
HHQKLVFFAED

SEQ ID NO: 5 - protein, 11 aa, synthetic construct
SGVDEAFFVLK

SEQ ID NO: 6 - protein, 12 aa, synthetic construct
KHHQKLVFFAED

SEQ ID NO: 7 - protein, 12 aa, synthetic construct
KSGVDEAFFVLK

SEQ ID NO: 8 - protein, 12 aa, synthetic construct
AHHQKLVFFAED

SEQ ID NO: 9 - protein, 12 aa, synthetic construct
ASGVDEAFFVLK

SEQ ID NO: 10 - protein, 4 aa, synthetic construct
KHHG

SEQ ID NO: 11 - protein, 4 aa, synthetic construct
AHHG

SEQ ID NO: 12 - protein, 12 aa, synthetic construct
KHHGKLVFFAED

SEQ ID NO: 13 - protein, 12 aa, synthetic construct
AHHGKLVFFAED

SEQ ID NO: 14 - protein, 12 aa, synthetic construct
KHHGDEAFFVLK

SEQ ID NO: 15 - protein, 12 aa, synthetic construct
AHHGDEAFFVLK

SEQ ID NO: 16 - protein, 12 aa, synthetic construct
KHHGKDEAFFVL

SEQ ID NO: 17 - protein, 12 aa, synthetic construct
AHHGKDEAFFVL

SEQ ID NO: 18 - protein, 12 aa, synthetic construct
KHHGADEAFFVL

SEQ ID NO: 19 - protein, 12 aa, synthetic construct
AHHGADEAFFVL

SEQ ID NO: 20 - protein, 9 aa, synthetic construct
CKLVFFAED